(19) United States
(12) Patent Application Publication
(10) Pub. No.: US 2004/0225206 A1
(43) Pub. Date: Nov. 11, 2004

(54) Non-invasive analyte measurement device having increased signal to noise ratios

(76) Inventor: Mikhail A. Kouchnir, Sunnyvale, CA (US)

Correspondence Address: MORRISON & FOERSTER LLP, 755 PAGE MILL RD, PALO ALTO, CA, 94304-1018, US

(21) Appl. No.: 10/434,963

(22) Filed: May 9, 2003

(51) Int. Cl. A61B 5/00

(52) U.S. Cl. 600/316; 600/322

(57) ABSTRACT

A non-invasive analyte measurement device having increased signal to noise ratios is disclosed herein. Typically, the glucose measurement device is self-normalizing in that it does not employ an independent reference sample in its operation. The device uses attenuated total reflection (ATR) infrared spectroscopy. The device is used on a fingertip and compares two specific regions of a measured infrared spectrum to determine the blood glucose level of the user. This device is suitable for monitoring glucose levels in the human body and is especially beneficial to users having diabetes mellitus. Moreover, the device utilizes a periodically modulated optical signal and at least one lock-in amplifier to correlate the signals to gain maximum sensitivity and an increased signal-to-noise ratio. The device and procedure can be used for other analyte materials which exhibit unique mid-IR signatures of the type described herein and that are found in appropriate regions of the outer skin.

### FIELD OF THE INVENTION

The present invention involves a non-invasive glucose measurement device and a process for determining blood glucose level in the human body using the device. More particularly, the inventive device uses attenuated total reflection (ATR) infrared spectroscopy suitable for monitoring glucose levels in the human body, and is especially beneficial to users having diabetes mellitus. The device and procedure may be used for other materials which exhibit unique mid-IR signatures of the type described below and that are found in appropriate regions of the outer skin.

### BACKGROUND OF THE INVENTION

The American Diabetes Association reports that nearly 6% of the population in the United States, a group of 16 million people, has diabetes. The Association further reports that diabetes is the seventh leading cause of death in the United States, contributing to nearly 200,000 deaths per year. Diabetes is a chronic disease having no cure. The complications of the disease include blindness, kidney disease, nerve disease, and heart disease, perhaps with stroke. Diabetes is said to be the leading cause of new cases of blindness in individuals in the range of ages between 20 and 74; from 12,000-24,000 people per year lose their sight because of diabetes. Diabetes is the leading cause of end-stage renal disease, accounting for nearly 40% of new cases. Nearly 60-70% of people with diabetes have mild to severe forms of diabetic nerve damage which, in severe forms, can lead to lower limb amputations. People with diabetes are 2-4 times more likely to have heart disease and to suffer strokes.

Diabetes is a disease in which the body does not produce or properly use insulin, a hormone needed to convert sugar, starches, and the like into energy. Although the cause of diabetes is not completely understood, genetics, environmental factors, and viral causes have been partially identified.

There are two major types of diabetes: Type I and Type II. Type I diabetes (formerly known as juvenile diabetes) is an autoimmune disease in which the body does not produce any insulin and most often occurs in young adults and children. People with Type I diabetes must take daily insulin injections to stay alive.

Type II diabetes is a metabolic disorder resulting from the body's inability to make enough, or properly to use, insulin. Type II diabetes accounts for 90-95% of diabetes. In the United States, Type II diabetes is nearing epidemic proportions, principally due to an increased number of older Americans and a greater prevalence of obesity and a sedentary lifestyle.

Insulin, in simple terms, is the hormone that unlocks the cells of the body, allowing glucose to enter those cells and feed them. Since, in diabetics, glucose cannot enter the cells, the glucose builds up in the blood and the body's cells literally starve to death.

Diabetics having Type I diabetes typically are required to self-administer insulin using, e.g., a syringe or a pin with needle and cartridge. Continuous subcutaneous insulin infusion via implanted pumps is also available. Insulin itself is typically obtained from pork pancreas or is made chemically identical to human insulin by recombinant DNA technology or by chemical modification of pork insulin. Although there are a variety of different insulins for rapid-, short-, intermediate-, and long-acting forms that may be used variously, separately or mixed in the same syringe, use of insulin for treatment of diabetes is not to be ignored.

It is highly recommended by the medical profession that insulin-using patients practice self-monitoring of blood glucose (SMBG). Based upon the level of glucose in the blood, individuals may make insulin dosage adjustments before injection. Adjustments are necessary since blood glucose levels vary day to day for a variety of reasons, e.g., exercise, stress, rates of food absorption, types of food, hormonal changes (pregnancy, puberty, etc.) and the like. Despite the importance of SMBG, several studies have found that the proportion of individuals who self-monitor at least once a day significantly declines with age. This decrease is likely due simply to the fact that the typical, most widely used, method of SMBG involves obtaining blood from a finger stick. Many patients consider obtaining blood to be significantly more painful than the self-administration of insulin.

There is a desire for a less invasive method of glucose measurement. Methods exist or are being developed for a minimally invasive glucose monitoring, which use body fluids other than blood (e.g., sweat or saliva), subcutaneous tissue, or blood measured less invasively. Sweat and saliva are relatively easy to obtain, but their glucose concentration appears to lag in time significantly behind that of blood glucose. Measures to increase sweating have been developed and seem to increase the timeliness of the sweat glucose measurement, however.

Subcutaneous glucose measurements seem to lag only a few minutes behind directly measured blood glucose and may actually be a better measurement of the critical values of glucose concentrations in the brain, muscle, and in other tissue. Glucose may be measured by non-invasive or minimally-invasive techniques, such as those making the skin or mucous membranes permeable to glucose or those placing a reporter molecule in the subcutaneous tissue. Needle-type sensors have been improved in accuracy, size, and stability and may be placed in the subcutaneous tissue or peripheral veins to monitor blood glucose with small instruments. See, “An Overview of Minimally Invasive Technologies”, Clin. Chem. 1992 September; 38(9):1596-1600.

Truly simple, non-invasive methods of measuring glucose are not commercially available.

U.S. Pat. No. 4,169,676 to Kaiser, shows a method for the use of ATR glucose measurement by placing the ATR plate directly against the skin and especially against the tongue. The procedure and device shown there uses a laser and determines the content of glucose in a specific living tissue sample by comparing the IR absorption of the measured material against the absorption of IR in a control solution by use of a reference prism. See, column 5, lines 31 et seq.

Swiss Patent No. 612,271, to Dr. Nils Kaiser, appears to be the Swiss patent corresponding to U.S. Pat. No. 4,169,676.

U.S. Pat. No. 4,655,255, to Dahne et al., describes an apparatus for non-invasively measuring the level of glucose in a blood stream or tissues of patients suspected to have diabetes. The method is photometric and uses light in the near-infrared region. Specifically, the procedure uses light in the 1,000 to 2,500 nm range. Dahne's device is jointly made up to two main sections, a light source and a detector section. They may be situated about a body part such as a finger. The desired near-infrared light is achieved by use of filters. The detector section is made up of a light-collecting integrating sphere or half-sphere leading to a means for detecting wavelengths in the near-infrared region. Dahne et al. goes to some lengths teaching away from the use of light in the infrared range having a wavelength greater than about 2.5 micrometers since those wavelengths are strongly absorbed by water and have very little penetration capability into living tissues containing glucose. That light is said not to be “readily useable to analyze body tissue volumes at depths exceeding a few microns or tens of microns.” Further, Dahne et al. specifically indicates that an ATR method which tries to circumvent the adverse consequences of the heat effect by using a total internal reflection technique is able only to investigate to tissue depths not exceeding about 10 micrometers, a depth which is considered by Dahne et al. to be “insufficient to obtain reliable glucose determination information.”

U.S. Pat. No. 5,028,787, to Rosenthal et al., describes a non-invasive glucose monitoring device using near-infrared light. The light is passed into the body in such a way that it passes through some blood-containing region. The so-transmitted or reflected light is then detected using an optical detector. The near-infrared light sources are preferably infrared emitting diodes (IRED). U.S. Pat. No. 5,086,229 is a continuation in part of U.S. Pat. No. 5,028,787.

U.S. Pat. No. 5,178,142, to Harjunmaa et al, teaches the use of a stabilized near-infrared radiation beam containing two alternating wavelengths in a device to determine a concentration of glucose or other constituents in a human or animal body. Interestingly, one of the transmitted IR signals is zeroed by variously tuning one of the wavelengths, changing the extracellular to intracellular fluid ratio of the tissue by varying the mechanical pressure on a tissue. Or, the ratio may be allowed to change as a result of natural pulsation, e.g., by heart rate. The alternating component of the transmitted beam is measured in the “change to fluid ratio” state. The amplitude of the varying alternating signal is detected and is said to represent glucose concentration or is taken to represent the difference in glucose concentration from a preset reference concentration.

U.S. Pat. No. 5,179,951 and its divisional, U.S. Pat. No. 5,115,133, to Knudson, show the application of infrared light for measuring the level of blood glucose in blood vessels in the tympanic membrane. The detected signal is detected, amplified, decoded, and, using a microprocessor, provided to a display device. The infrared detector (No. 30 in the drawings) is said simply to be a “photo diode and distance signal detector” which preferably includes “means for detecting the temperature of the volume in the ear between the detector and the ear's tympanic membrane.” Little else is said about the constituency of that detector.

U.S. Pat. No. 5,433,197, to Stark, describes a non-invasive glucose sensor. The sensor operates in the following fashion. A near-infrared radiation is passed into the eye through the cornea and the aqueous humor, reflected from the iris or the lens surface, and then passed out through the aqueous humor and cornea. The reflected radiation is collected and detected by a near-infrared sensor which measures the reflected energy in one or more specific wavelength bands. Comparison of the reflected energy with the source energy is said to provide a measure of the spectral absorption by the eye components. In particular, it is said that the level of glucose in the aqueous humor is a function of the level of glucose in the blood. It is said in Stark that the measured glucose concentration in the aqueous humor tracks that of the blood by a fairly short time, e.g., about 10 minutes. The detector used is preferably a photodiode detector of silicon or InGaAs. The infrared source is said preferably to be an LED, with a refraction grating so that the light of a narrow wavelength band, typically 10 to 20 nanometers wide, passes through the exit slit. The light is in the near-infrared range. The use of infrared regions below 1400 nanometers and in the region between 1550 and 1750 nanometers is suggested.

U.S. Pat. No. 5,267,152, to Yang et al., shows a non-invasive method and device for measuring glucose concentration. The method and apparatus uses near-infrared radiation, specifically with a wavelength of 1.3 micrometers to 1.8 micrometers from a semiconductor diode laser. The procedure is said to be that the light is then transmitted down through the skin to the blood vessel where light interacts with various components of the blood and is then diffusively reflected by the blood back through the skin for measurement.

Similarly, U.S. Pat. No. 5,313,941, to Braig et al., suggests a procedure and apparatus for monitoring glucose or ethanol and other blood constituents in a non-invasive fashion. The measurements are made by monitoring absorption of certain constituents in the longer infrared wavelength region. The long wavelength infrared energy is passed through the finger or other vascularized appendage. The infrared light passing through the finger is measured. The infrared source is pulsed to prevent burning or other patient discomfort. The bursts are also synchronized with the heartbeat so that only two pulses of infrared light are sent through the finger per heartbeat. The detected signals are then analyzed for glucose and other blood constituent information.

U.S. Pat. No. 5,398,681, to Kuperschmidt, shows a device which is said to be a pocket-type apparatus for measurement of blood glucose using a polarized-modulated laser beam. The laser light is introduced into a finger or ear lobe and the phase difference between a reference signal and the measurement signal is measured and processed to formulate and calculate a blood glucose concentration which is then displayed.

U.S. Pat. No. 6,001,067 shows an implantable device suitable for glucose monitoring. It utilizes a membrane which is in contact with a thin electrolyte phase, which in turn is covered by an enzyme-containing membrane, e.g., glucose oxidase in a polymer system. Sensors are positioned in such a way that they measure the electro-chemical reaction of the glucose within the membranes. That information is then passed to the desired source.

None of the cited references suggests the device and method of using this device described and claimed below.

### BRIEF SUMMARY OF THE INVENTION

A glucose level measurement device utilizing infrared attenuated total reflection (IR-ATR) spectroscopy may typically comprise an IR source for emitting an IR beam into the ATR plate, IR sensors for simultaneously measuring absorbance of at least two specific regions of the IR spectrum, i.e., a “referencing wavelength” and a “measuring wavelength”, and lock-in amplifiers to “track” the measured signals. The IR source preferably emits IR radiation at least in the region of the referencing wavelength and the measuring wavelength. For glucose, the referencing wavelength is between about 8.25 micrometers and about 8.75 micrometers and the measuring wavelength is between about 9.50 micrometers and about 10.00 micrometers.

The IR sources may be broadband IR sources, non-laser sources, or two or more selected wavelength lasers. The IR sources may also be electrically connected to a driver or modulator which may be used to modulate the intensity of the IR optical beam. The intensity of the optical signal may be modulated in a variety of patterns, e.g., sinusoidal, saw tooth, square wave, etc., so long as the signal is continuously and periodically modulated. The modulator is also connected to a pair of lock-in amplifiers and may be used to provide the reference signal to the lock-in amplifiers. By periodically modulating the intensity of the emitted beam at some frequency, the modulator also functions to shift the spectrum of the emitted IR beam to a higher frequency with respect to an unmodulated beam. Shifting the optical signals to a higher frequency reduces the system noise, which is normally highest at lower frequencies.

Each of the IR sensors may be in communication with a corresponding lock-in amplifier. Because the signals detected by the IR sensors are in a fixed phase relationship with the emitted beam due to the referenced signal provided by the modulator, the device is able to “track” changes in the frequency of the detected signal. As a result, the signals detected by the IR sensors have a maximum sensitivity and allows for a small detection bandwidth as the lock-in amplifiers act as narrow band filters. The signal-to-noise ratio may thus be improved significantly by employing the lock-in amplifiers as frequency drift is eliminated. The number of lock-in amplifiers utilized may depend upon the number of sensors used to detect the optical signals.

Other analyte materials which have both referencing wavelengths and measuring wavelengths as are described in more detail below and that preferably are found in the outer regions of the skin may be measured using the inventive devices and procedures described herein.

The ATR plate is configured to permit multiple internal reflections, perhaps 3-15 internal reflections or more, against the measurement surface prior to measurement by the IR sensors. Typically the IR beam emitted from the ATR plate is split for the IR sensors using a beam splitter or equivalent optical device. Once the split beams are measured by the IR sensors, the resulting signals are then transformed using processors, e.g., analog comparators or digital computers, into readable or displayable values. The measured and/or processed signals may also be stored in a memory unit for historical comparison or for retrieval at a later time.

### BRIEF DESCRIPTION OF THE DRAWINGS

FIGS. 1A, 1B, 1C, and 1D show a side view of various ATR plates and their general operation.

FIG. 2 shows an IR spectrum of d-glucose.

FIG. 3 shows a schematic layout of the optics and electronics of the inventive device.

FIGS. 4A and 4B show alternative variations for detecting the reflected light with the measuring device.

FIG. 5 shows a variation of the measuring device with an optional pressure maintaining component.

FIG. 6 shows a graph of pressure on the ATR crystal versus IR value.

FIG. 7 shows a graph using a transmittance trough as the referencing wavelength.

### DETAILED DESCRIPTION OF THE INVENTION

The device in this invention uses infrared (“IR”) attenuated total reflectance (“ATR”) spectroscopy to detect and ultimately to determine the level of a selected analyte, preferably blood glucose, in the human body. Preferably, the inventive device uses an ATR procedure in which the size and configuration of the crystal permits a number of internal reflections before the beam is allowed to exit the crystal with its measured information. A detailed description of the device and examples of use are described in U.S. Pat. No. 6,424,851 which is incorporated herein by reference in its entirety.

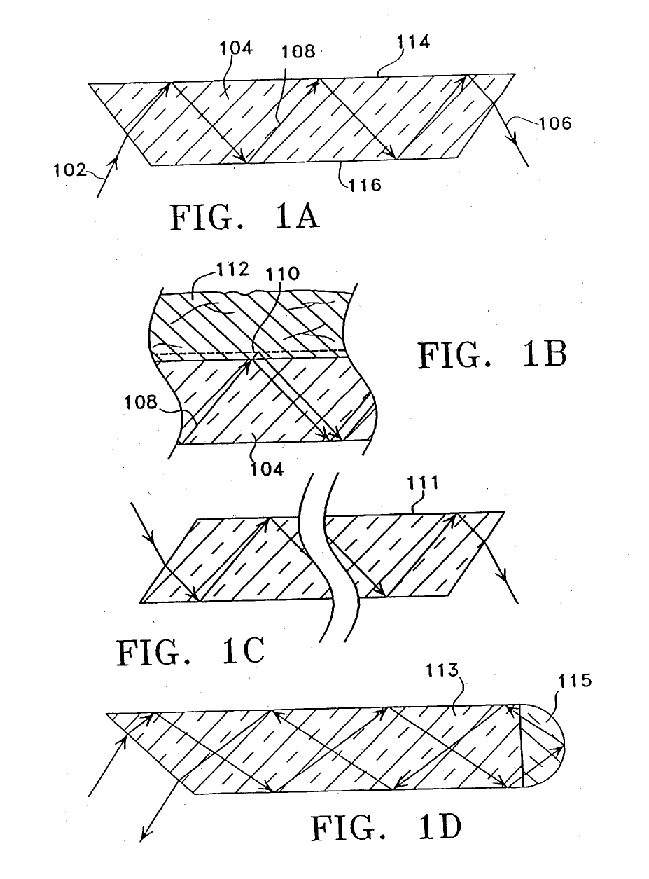

In general, as shown in FIGS. 1A and 1B, when an infrared beam 102 is incident on the upper surface 114 of the ATR crystal 104—or ATR plate—at an angle which exceeds a critical angle Θ_{C}, the beam 102 will be completely totally reflected within crystal 104, e.g., a ZnSe crystal ATR plate. Each reflection of the beam within the ATR plate, and specifically against the upper surface 114, provides a bit more information about the composition of the sample 112 resting against that upper surface 114. The more numerous the reflections, and the greater the penetration depth of the reflection, the higher is the quality of the information. The incident beam 102 becomes reflected beam 106 as it exits crystal 104 as shown in FIG. 1A. Higher refractive index materials are typically chosen for the ATR crystal to minimize the critical angle. The critical angle is a function of the refractive indices of both the sample and the ATR crystal and is defined as:

$${Θ}_{c} = {sin}^{- 1} ( \frac{{n}_{2}}{{n}_{1}} )$$

Here, n₁ is the refractive index of the ATR crystal and n₂ is the refractive index of the sample.

Throughout this specification, we refer to wavelength measures as specific values. It should be understood that we intend those values to be bands or ranges of values, typically with a tolerance of +/−0.20 micron; however, the tolerance range may be higher or lower depending upon the application and desired results.

As shown in FIG. 1B, the internally reflected beam 108 includes an evanescent wave 110 which penetrates a short distance into sample 112 over a wide wavelength range. In those regions of the IR spectrum in which the sample absorbs IR, some portion of the light does not return to the sensor. It is these regions of IR absorbance which provide information, in this inventive device, for quantification of the glucose level.

We have found that the mid-IR spectrum does not penetrate into the skin to an appreciable level. Specifically, the skin is made up of a number of layers: the outermost—the stratum corneum—is a layer substantially free of cholesterol, water, gamma globulin, albumin, and blood. It is a shallow outer region covering the stratum granulosum, the stratum spinosum, and the basal layer. The area between the basal layer to the outside is not vascularized. It is unlikely that any layer other than the stratum corneum is traversed by the mid-IR light involved in this inventive device. Although we do not wish to be bound by theory, it is likely that the eccrine or sweat glands transport the glucose to the outer skin layers for measurement and analysis by our inventions.

We prefer the use of higher refractive index crystals such as zinc selenide, zinc sulfide, diamond, germanium, and silicon as the ATR plate. The index of refraction of the ATR plate 104 should be significantly higher than that of the sample 112.

Further, the ATR crystal 104 shown in FIG. 1A is shown to be trapezoidal and having an upper surface 114 for contact with the sample, which sample, in this case, is skin from a living human body. However, this shape is only for the purposes of mechanical convenience and ease of application into a working commercial device. Other shapes, in particular, a parallelogram 111 such as shown in FIG. 1C and the reflective crystal 113 shown in FIG. 1D having mirrored end 115, are also quite suitable for this inventive device should the designer so require. The mirrored reflective crystal 113 has the advantage of, and perhaps the detriment of having both an IR source and the IR sensors at the same end of the crystal.

It is generally essential that the ATR crystal or plate 104 have a sample or upper surface 114 which is essentially parallel to the lower surface 116. In general, the ATR plate 104 is preferably configured and utilized so that the product of the practical number of internal reflections of internal reflected beam 108 and the skin penetration per reflection of this product is maximized. When maximizing this product, called the effective pathlength (EPL), the information level in beam 106 as it leaves ATR plate 104 is significantly higher. Further, the higher the value of the index of refraction, n₂, of the ATR plate 104, the higher is the number of internal reflections. The sensitivity of the IR sensors also need not be as high when the EPL is maximized. We consider the number of total reflections within the crystal to be preferably from 3-15 or more for adequate results.

We have surprisingly found that a glucose measuring device made according to this invention is quite effective on the human skin of the hands and fingers. We have found that the glucose concentration as measured by the inventive devices correlates very closely with the glucose concentration determined by a direct determination from a blood sample. The glucose level as measured by the inventive device also is surprisingly found closely to track the glucose level of blood in time as well. This is surprising in that the IR beam likely passes into the skin, i.e., the stratum corneum, for only a few microns. It is unlikely in a fingertip that any blood is crossed by that light path. As discussed above, the stratum corneum is the outer layer of skin and is substantially unvascularized. The stratum corneum is the final outer product of epidermal differentiation or keratinization. It is made up of a number of closely packed layers of flattened polyhedral corneocytes (also known as squames). These cells overlap and interlock with neighboring cells by ridges and grooves. In the thin skin of the human body, this layer may be only a few cells deep, but in thicker skin, such as may be found on the toes and feet, it may be more than 50 cells deep. The plasma membrane of the corneocyte appears thickened compared with that of keratinocytes in the lower layers of the skin, but this apparent deposition of a dense marginal band formed by stabilization of a soluble precursor, involucrin, just below the stratum corneum.

It may sometimes be necessary to clean the skin exterior prior before sampling to remove extraneous glucose from the skin surface. Examples of cleaning kits and various components which may be used with the cleaning kits are described in further detail in U.S. Pat. No. 6,362,144 and U.S. patent application Ser. No. 10/358,880 filed Feb. 4, 2003, each of which is incorporated herein by reference in its entirety.

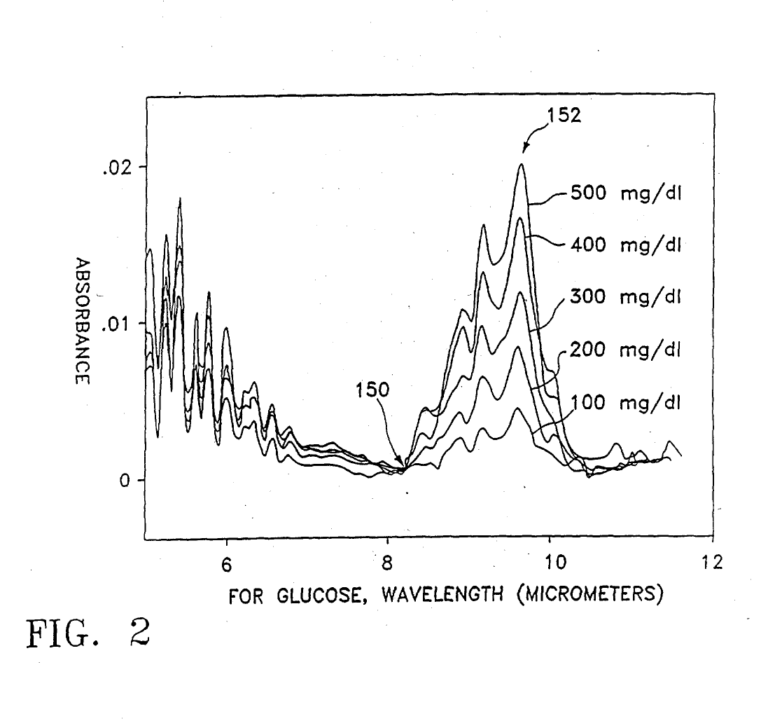

Additionally, the inventive device can be highly simplified compared to other known devices in that the device can be “self-normalizing” due to the specifics of the IR signature of glucose. FIG. 2 shows the IR absorbance spectra of d-glucose. The family of curves there shows that in certain regions of the IR spectrum, there is a correlation between absorbance and the concentration of glucose. Further, there is a region in which the absorbance is not at all dependent upon the concentration of glucose. Our device, in its preferable method of use, uses these two regions of the IR spectra. These regions are in the so-called mid-IR range, i.e., wavelengths between 2.5 and 14 micrometers. In particular, the “referencing wavelength” point is just above 8 micrometers 150, e.g., 8.25 to 8.75 micrometers, and the pronounced peaks 152 at the region between about 9.50 and 10.00 micrometers is used as a “measuring wavelength”. The family of peaks 152 may be used to determine the desired glucose concentration.

Use of the two noted IR regions is also particularly suitable since other components typically found in the skin, e.g., water, cholesterol, etc., do not cause significant measurement error when using the method described herein.

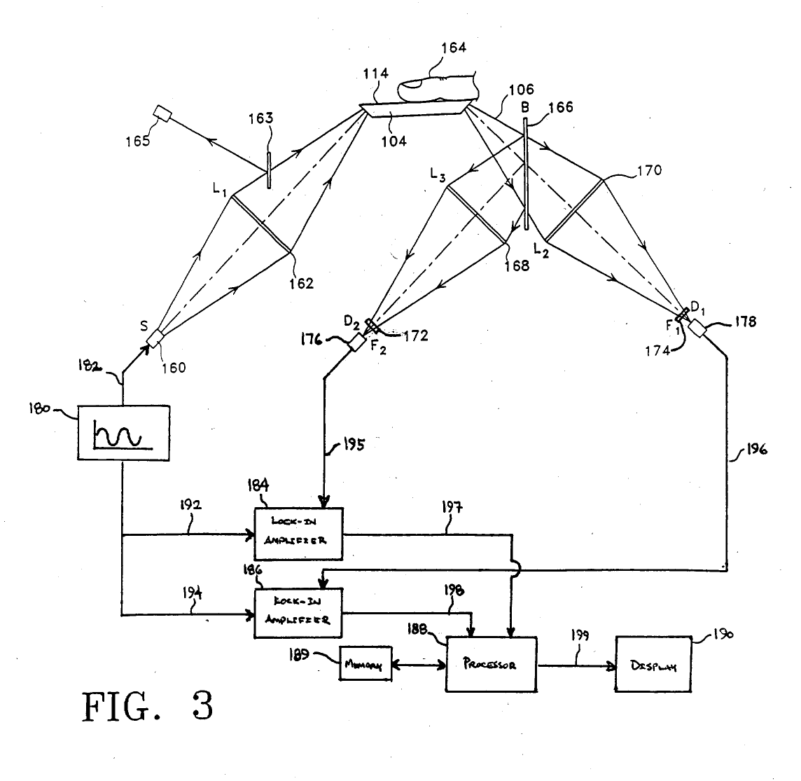

FIG. 3 shows an optical and electrical schematic of a desired variation of the inventive device. ATR crystal 104 with sample side 114 is shown and IR source 160 is provided. IR source 160 may be any of a variety of different kinds of sources. It may be a broadband IR source, one having radiant temperatures of 300° C. to 800° C., or a pair of IR lasers selected for the two regions of measurement discussed above, or other suitably emitted or filtered IR light sources. A single laser may not be a preferred light source in that a laser is a single wavelength source and the preferred operation of this device requires light sources simultaneously emitting two IR wavelengths. Lens 162, for focusing light from IR source 160 into ATR plate 104, is also shown. An additional mirror 163 may optionally be included to intercept a portion of the beam before it enters the ATR plate 104 and to measure the strength of that beam in IR sensor 165. Measurement of that incident light strength (during normalization and during the sample measurement) can be used to assure that any changes in that value can be compensated for.

IR source 160 may be electrically connected via line 182 to a driver or modulator 180, which generates a reference signal and modulates the IR optical beam. The optical signal may be modulated in a variety of patterns, e.g., sinusoidal, saw tooth, square wave, etc., so long as the signal is continuously and periodically modulated. Modulator 180 is also electrically connected to a pair of lock-in amplifiers 184, 186 via lines 192, 194, respectively, and may be used to provide the reference signal to the lock-in amplifiers 184, 186, as described in further detail below. By periodically modulating the emitted beam, the modulator 180 also functions to shift the spectrum of the emitted beam 106 to a higher frequency. Shifting the optical signals to a higher frequency reduces the system noise, which is normally highest at lower frequencies.

The modulated light then passes into ATR plate 104 for contact with body part 164, shown in this instance to be the desired finger. The reflected beam 106 exits ATR plate 104 and may be split using beam splitter 166. Beam splitter 166 simply transmits some portion of the light through the splitter and reflects the remainder. The two beams may then be passed through, respectively, lenses 168 and 170. The so-focused beams are then passed to a pair of sensors which are specifically selected for detecting and measuring the magnitude of the two beams in the selected IR regions. Generally, the sensors will be made up of filters 172 and 174 with light sensors 176 and 178 behind. Generally, one of the filters 172, 174 will be in the region of the referencing wavelength and the other will be in that of the measuring wavelength.

Each of the light sensors 176 and 178 may be in electrical communication with a corresponding lock-in amplifier 184 and 186, e.g., light sensor 176 may be connected via line 195 to an input of lock-in amplifier 184 and light sensor 178 may be connected via line 196 to an input of lock-in amplifier 186. The reflected beam 106, and the signals detected by light sensors 176 and 178, are in a fixed phase relationship with the emitted beam due to the referenced signal provided by the modulator 180. The use of this common reference signal helps to ensure that the device “tracks” changes in the frequency of the detected signal. As a result, the light signals detected by light sensors 176 and 178 have a maximum sensitivity and allows for a small detection bandwidth as the lock-in amplifiers 184, 186 act as narrow band filters. Moreover, the signal-to-noise ratio is improved significantly by employing the lock-in amplifiers 184, 186 as frequency drift is eliminated. The number of lock-in amplifiers utilized may depend upon the number of sensors used to detect the optical signals.

The signals received by the lock-in amplifiers 184, 186 may then be transmitted via lines 197, 198, respectively, to a processor 188, e.g., a computer, which may be used to determine the measured glucose or analyte levels. The results of the measurements may then be transmitted via line 199 to display unit 190, which may be used to display the information in a variety of forms, e.g., numerically, graphically, etc. The modulated nature of the signals may also allow the device to process and display measurements in real-time or to store the data for later access. The processor 188 may thus be in electrical communication with a memory storage unit 189, which may be used to store a history of measured data. The continuous modulation of the signals also allows for the continuous recording and storage of the measurements within memory storage unit 189. The storage unit 189 may additionally allow a user to access the stored history of measurements at any point in time for display on either the display unit 190 or allow for the downloading of this data onto another medium.

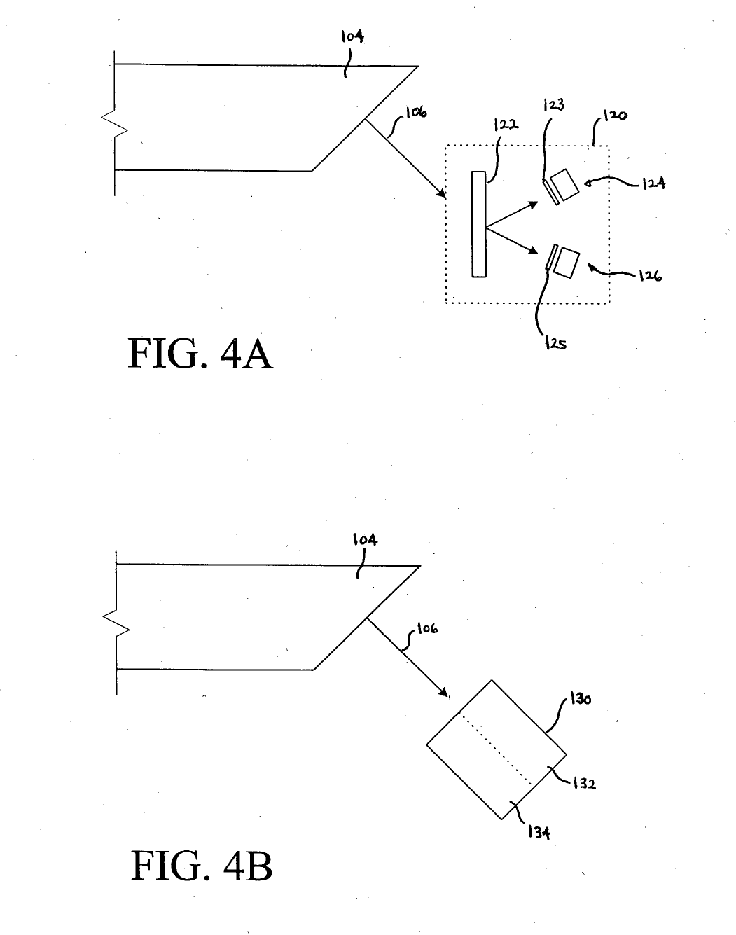

FIGS. 4A and 4B show alternative variations of light sensors which may be used to detect the reflected beam 106. As shown in FIG. 4A, sensor assembly 120 may be assembled in a single integrated unit. Within this unit 120, the reflected beam 106 may be incident upon a beam splitter 122. The beam splitter 122 may split the beam into two separate beams, which may be incident upon filter 123 with sensor 124 and filter 125 with sensor 126. Another variation may be seen in FIG. 4B, which shows the reflected beam 106 incident upon a single sensor assembly 130, which omits a beam splitter. Instead, sensors 132, 134 may be aligned adjacent to one another such that the reflected beam 106 is incident upon both sensors without the need for a beam splitter.

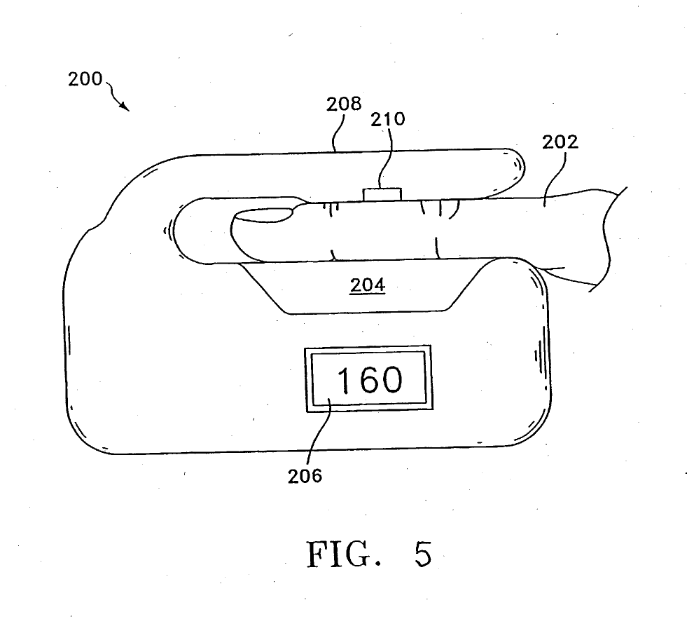

FIG. 5 shows perhaps a variation of this device 200 showing the finger of the user 202 over the ATR plate 204 with a display 206. Further shown in this desirable variation 200 is a pressure maintaining component 208. We have found that is very highly desirable to maintain a minimum threshold pressure on the body part which is to be used as the area to be measured. Generally, a variance in the pressure does not shift the position of the detected IR spectra, but it may affect the sensitivity of the overall device. Although it is possible to teach the user to press hard enough on the device to reach the minimum threshold pressure, we have determined for each design of the device it is much more appropriate that the design of a particular variation of the inventive device be designed with a specific sample pressure in mind. The appropriate pressure will vary with, e.g., the size of the ATR plate and the like. A constant pressure above that minimum threshold value is most desired.

The variation shown in FIG. 5 uses a simple component arm 208 to maintain pressure of the finger 202 on ATR plate 204. Other variations within the scope of this invention may include clamps and the like.

It should be apparent that once an appropriate pressure is determined for a specific design, the inventive device may include a pressure sensor, e.g., 210 as is shown in FIG. 5, to measure adherence to that minimum pressure. Pressure sensor 210 may alternatively be placed beneath ATR plate 204. It is envisioned that normally a pressure sensor such as 210 would provide an output to the user indicating a “no-go/go” type of signal.

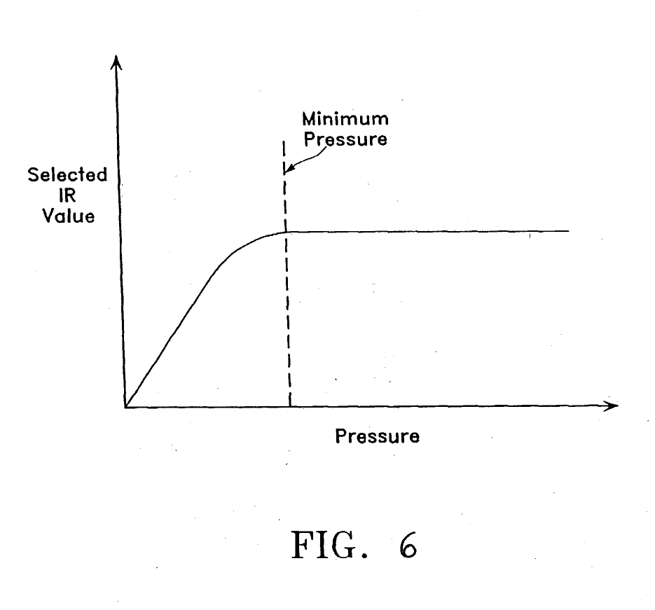

Further, as shown in FIG. 6, the appropriate pressure may be achieved when using our device, simply by increasing the pressure of the body part on the ATR crystal surface until the pressure is within a selected pressure window (i.e., greater than a minimum pressure and lower than a maximum pressure), at which time the device obtains the desired measurement.

In general, the inventive device described above may be used in the following manner: a skin surface on a human being, for instance, the skin of the finger, is placed on the ATR plate. The skin surface is radiated with an IR beam having components at least in the two IR regions we describe above as the “referencing wavelength” and the “measuring wavelength.” The beam which ultimately is reflected out of the ATR plate then contains information indicative of the blood glucose level in the user. As noted above, it is also desirable to maintain that skin surface on the ATR plate at a relatively constant pressure that is typically above a selected minimum pressure. This may be done manually or by measuring and maintaining the pressure or monitoring the constancy of a selected IR value.

Typically, the beam leaving the ATR plate may be then focused each onto its own IR sensor. Each such IR sensor has a specific filter. This is to say that, for instance, one IR sensor may have a filter which removes all light which is not in the region of the referencing wavelength and the other IR sensor would have a filter which remove all wavelengths other than those in the region of the measuring wavelength. As noted above, for glucose, the referencing wavelength is typically in the range of about 8.25 to 8.75 micrometers. For glucose, the measuring wavelength is typically between about 9.5 and 10.0 micrometers.

Other analyte materials which have both referencing wavelengths and measuring wavelengths in the mid-IR range and that are found in the outer regions of the skin may also be measured using the inventive devices and procedures described herein.

Respective signals may be compared using analog or digital computer devices, e.g., processor 188. The signals are then used to calculate analyte values such as blood glucose concentration using various stored calibration values, typically those which are discussed below. The resulting calculated values may then be displayed.

As noted above, it is also desirable both to clean the plate before use and to clean the exterior surface of the skin to be sampled. Again, we have found, for instance in the early morning that the exterior skin is highly loaded with glucose which is easily removed preferably by using the skin preparation kit, or, less preferably, by washing the hands. Reproducible and accurate glucose measurements may then be had in a short period, e.g., 10 minutes or less, after cleaning the area of the skin to be measured.

We also note that, depending upon the design of a specific variation of a device made according to the invention, periodic at least an initial calibration of the device, using typical blood sample glucose determinations, may be necessary or desirable.

Determination of blood glucose level from the information provided in the IR spectra is straightforward. A baseline is first determined by measuring the level of infrared absorbance at the measuring and referencing wavelengths, without a sample being present on the sample plate. The skin is then placed in contact with the ATR plate and the two specified absorbance values are again measured. Using these four values, the following calculation is then made.

$${A}_{1} = ln ( \frac{{T}_{01}}{{T}_{1}} ) = {A}_{g1} + {A}_{b1}$$

(Absorbance at referencing spectral band.)

$${A}_{2} = ln ( \frac{{T}_{02}}{{T}_{2}} ) = {A}_{g2} + {A}_{b2}$$

(Absorbance at measuring spectral band.)

where:

T₀₁=measured value at reference spectral band w/o sample

T₀₂=measured value at measuring spectral band w/o sample

T₁=measured value at reference spectral band w/ sample

T₂=measured value at measuring spectral band w/ sample

A_{g1}=absorbance of glucose at reference spectral band

A_{g2}=absorbance of glucose at measuring spectral band

A_{b1}=absorbance of background at reference spectral band

A_{b2}=absorbance of background at measuring spectral band

d=effective path length through the sample.

a₂=specific absorptivity at measuring spectral band

k=calibration constant for the device

C_{g}=measured concentration of glucose

Since the background base values are approximately equal (i.e., A_{b1}=A_{b2}) and A_{g1}=0, then:

A₂−A₁=A_{g2}=a₂dC_{g}

and

C_{g}=k(A₂−A₁)

The value of C_{g} is the desired result of this procedure.

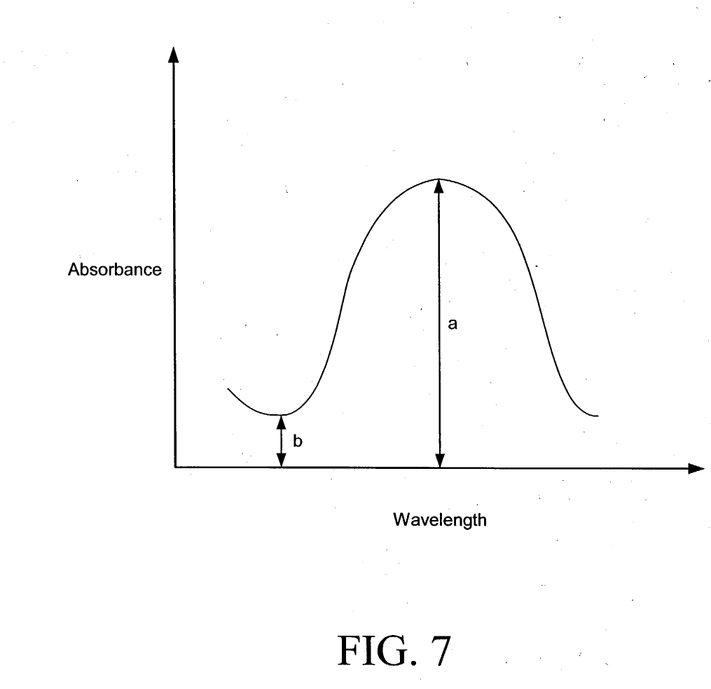

Similarly, FIG. 7 shows a graph in which the value of the analyte is assessed using similar calculations but in which the “referencing wavelength” is an absorbance trough (“b”) unaffected by the concentration of the analyte. The “measuring wavelength” peak (“a”) is measured against a baseline.

This invention has been described and specific examples of the invention have been portrayed. The use of those specifics is not intended to limit the invention in any way. Additionally, to the extent there are variations of the invention with are within the spirit of the disclosure and yet are equivalent to the inventions found in the claims, it is our intent that this patent will cover those variations as well.

I claim:

1. An analyte level measurement device for measuring an analyte level in a body by contacting a skin surface, comprising:
an infrared source for emitting an IR beam into an ATR plate, the IR beam having components at least in the region of a referencing wavelength and a measuring wavelength;
at least two IR sensors for simultaneously measuring absorbance of at least the referencing wavelength and the measuring wavelength; and
at least one lock-in amplifier in communication with a corresponding IR sensor, the at least one lock-in amplifier being adapted to correlate the infrared source with an output signal from the IR sensors.

2. The device of claim 1 wherein the ATR plate has a measurement surface for contact with the skin surface and for directing the IR beam against the skin surface.

3. The device of claim 1 further comprising a processor for determining the analyte level using a measured absorbance of the skin surface.

4. The device of claim 3 wherein the processor comprises a comparator for comparing the measuring wavelength to the referencing wavelength and providing a signal indicative of blood glucose concentration.

5. The device of claim 1 further comprising a modulator for periodically modulating an intensity of the IR beam and maintaining a fixed relationship with the at least one lock-in amplifier.

6. The device of claim 5 wherein the fixed relationship is a fixed phase relationship.

7. The device of claim 5 wherein the IR beam intensity is modulated in a pattern selected from the group consisting of sinusoidal, saw tooth, and square wave.

8. The device of claim 1 further comprising a memory unit for storing parameters of the measured absorbance.

9. The device of claim 1 further comprising a display for indicating a processed result from the measured absorbance.

10. The device of claim 1 wherein the ATR plate is configured to permit multiple internal reflections therewithin prior to measuring the absorbance.

11. The device of claim 1 wherein the analyte comprises glucose and the referencing wavelength is between about 8.25 μm and 8.75 μm.

12. The device of claim 1 wherein the analyte comprises glucose and the measuring wavelength is between about 9.50 μm and 10.00 μm.

13. The device of claim 1 further comprising a beam splitter positioned between the ATR plate and the at least two IR sensors.

14. The device of claim 1 wherein each of the at least two IR sensors is in communication with a corresponding lock-in amplifier.

15. A method for determining an analyte level in a body with an analyte measurement device, comprising:
contacting a skin surface on the body with an ATR plate in the analyte measurement device, the ATR plate having a surface for contact with the skin surface;
irradiating the skin surface with a periodically modulated IR beam having components at least in a region of a referencing wavelength and a measuring wavelength through the ATR plate to produce a reflected IR beam indicative of the analyte level in the body; and
correlating the referencing wavelength and the measuring wavelength components in the reflected IR beam with the periodically modulated IR beam so as to detect the analyte level.

16. The method of claim 15 further comprising maintaining the skin surface against the ATR plate at an adequate pressure.

17. The method of claim 15 further comprising normalizing the analyte measurement device by simultaneously detecting and quantifying the referencing wavelength and the measuring wavelength components in the reflected IR beam prior to contacting the skin surface on the body with the ATR plate.

18. The method of claim 15 where the periodically modulated IR beam is modulated in a pattern selected from the group consisting of sinusoidal, saw tooth, and square wave.

19. The method of claim 15 where correlating the referencing wavelength and the measuring wavelength components comprises correlating the components via at least one lock-in amplifier.

20. The method of claim 15 further comprising storing parameters of the analyte level in a memory unit.

21. The method of claim 15 further comprising displaying the analyte level.

22. The method of claim 15 wherein the referencing wavelength is between about 8.25 μm and about 8.75 μm.

23. The method of claim 15 wherein the measuring wavelength is between about 9.50 μm and about 10.00 μm.

24. The method of claim 15 further comprising splitting the reflected beam to form two beams and introducing the two beams each to one of at least two IR sensors.
